(19) United States
(12) Patent Application Publication
(10) Pub. No.: US 2024/0008784 A1
(43) Pub. Date: Jan. 11, 2024

(54) System and Method for Prevention, Diagnosis, and Treatment of Health Conditions

(71) Applicant: Innsightful, Inc., Ann Arbor, MI (US)

(72) Inventors: Farzad Ehsani, Sunnyvale, CA (US); Demitrios L. Master, Cupertino, CA (US)

(21) Appl. No.: 18/039,743

(22) PCT Filed: Nov. 30, 2021
(86) PCT No.: PCT/US2021/061136

§ 371 Date: Jun. 1, 2023

Related U.S. Application Data
(60) Provisional application No. 63/120,251, filed on Dec. 2, 2020.

(51) Int. Cl. A61B 5/16; G16H 50/20; G16H 20/70; A61B 5/0205; A61B 5/00

(52) U.S. Cl. CPC A61B 5/165; G16H 50/20; G16H 20/70; A61B 5/0205; A61B 5/486; A61B 5/7435; A61B 5/7455

(57) ABSTRACT

A system for management of health conditions, such as mental health conditions, using a biosensor configured to monitor one or more biological properties of an individual and providing feedback and information based on data in real-time. A corresponding method, performed by one or more components of the system, may include monitoring abiological property of the individual, identifying an irregularity as a symptom of the health condition, and intervening with the individual to alleviate the symptom. The method may further include determining an efficacy of the intervention and adapting the method to improve the intervention. The system may be configured to predict a future symptom and preventatively intervene.

### TECHNICAL FIELD

The present invention relates to an artificial intelligence (AI) system for monitoring and managing one or more health conditions of an individual. More particularly, the system includes a biosensor which monitors a biological property of the individual and produces one or more signals that relate to the biological property, processing data that pertains to the one or more signals and identifies one or more irregularities as corresponding with a symptom of a health condition, such as a mental health condition, intervenes with the individual to alleviate the symptom, and may be adapted to improve the intervention and may intervene to prevent or mitigate future symptoms based on learned data.

### BACKGROUND

Many individuals, both in the U.S. and throughout the world, suffer from adverse mental health conditions. Currently, 70.5% of college students report their academic performance is affected by stress; 53.1% are affected by anxiety, and 34.5% by depression. Student use of college counseling centers increased by an average of 30-40% from 2009 to 2015. Many college campuses have no on-campus psychiatric support, and if they do, wait times are extremely long, and many students end up in crisis. In addition, it is estimated that 49% of the general population has a history of anxiety, depression, substance abuse, or some degree of all three. The most common mental health issues among college students are anxiety, depression, relationship issues, and thoughts of suicide. However, and unfortunately, approximately only half receive treatment, which may be due to significant stigma around mental health and may also point to significant issues with the current mental healthcare treatment model.

Existing solutions for monitoring and treating mental health conditions do not adequately meet the growing demand for these services. Limited availability of many therapists and psychiatrists leads many patients to forego treatment altogether or even experience significant mental health crises while waiting to see a health professional. Additional factors, such as transportation cost or availability, and lack of acceptable health insurance, complicate traditional treatment approaches. The effect is a high healthcare and economic burden, on both caretakers and patients. As a result, many patients suffer for extended periods of time without receiving effective treatment.

The prevalence of mobile computing devices, such as smartphones, tablets, and wearable biosensors, has led to different types of personal monitoring for achievement of recreational fitness, sports, and leisurely goals. Many biosensors are configured to detect heart rate as a fitness parameter, but this class of technology has yet to be fully realized with respect to its potential to monitor, diagnose, treat, and prevent adverse health events, particularly mental health events. Despite the modern availability of technology, there is a substantial time delay between onset of symptoms and receipt of treatment, often measured in years, which needs to be addressed.

Accordingly, there exists a need in the art for real-time automated monitoring, and intervening, with patients for the prevention, diagnosis, and treatment of health conditions, particularly mental health conditions. The present specification addresses this unmet need.

### SUMMARY

The present specification provides a system, method, and machine-readable media for improved management of one or more health conditions, including communicable and non-communicable diseases and disorders, and including any condition that may be detrimental to one or more individuals using a biosensing device typically worn by the user. By way of example only, the health condition may be a viral infection (e.g., influenza, COVID-19), or may be a mental health condition, including but not limited to anxiety and depression. [symbol] mental health conditions with one or more non-mental health conditions.

In one aspect, the present system and methods provides a system for management of a health condition, comprising one or more devices configured to monitor a biological property of an individual, identify an irregularity of the biological property as a symptom of the health condition, and intervene with the individual to alleviate the symptom. The system is useful for automating management of health conditions, including mental health conditions, in day-to-day micro-environments that are generally inaccessible to existing healthcare services. In the case of mental health care, the intervention may include an appropriate guided therapy, such as cognitive behavioral therapy (CBT). In the case of various types of health care, whether mental or physical in nature, the intervention may be comprised of a recommendation to take a particular course of action, such as self-administering one or more medications or consulting with a healthcare professional. If the healthcare professional is to be consulted, the system may facilitate the consultation by connecting the individual to the healthcare professional.

A system operated by a user, the user having a mobile device, the system includes at least one hardware processor, a biosensor device operably connected to the mobile device, the biosensor device wearable by the user, and the at least one hardware processor performing a series of steps to manage health condition, the series of steps including monitoring for an irregularity, identifying a symptom, based on the symptom the system will report the status of the symptom to the user, offering an intervention based on an evaluation of the symptom, providing the intervention to the user, and determining an efficacy of the intervention, and improving the monitoring of the symptom of the user based on the learned information.

In some embodiments, the operation of the system further includes the step of adapting the operations to improve the efficacy of the intervening with the individual. In other embodiments, the determining the efficacy of the intervening with the individual comprises querying the individual with a dialog operation of the system.

The system may include a networked computational server of the system comprises the at least one hardware processor, wherein the system comprises a biosensor device operably connected to the networked computational server wherein the mobile device may be operably connected to the networked computational server and the biosensor device.

In some embodiments, the operations may further include reporting a status of the individual to a third party.

Further, the biosensor may be configured to produce one or more signals that relate to the biological property, wherein the biological property is selected from a group consisting essentially of: a heart rate (HR), a heart rate variability (HRV), an oxygen saturation (Sp02), an electrodermal activity (EDA), a breathing rate (BR), a movement, a body temperature (BT), and any combination thereof.

In some embodiments, the system may include a camera system to image the individual and produce one or more signals that relate to the biological property based on a processing of one or more images of the individual.

In some embodiments, the intervention to alleviate the symptom comprises a therapy. The therapy may be selected from a group consisting essentially of engaging in cognitive-behavioral therapy (CBT), engaging in dialectical behavior therapy (DBT), engaging in acceptance and commitment therapy (ACT), engaging in a positive activity, engaging in a breathing exercise, and any combination thereof.

In another embodiment, a system operated by a user is provided, the user having a mobile device, the system including at least one hardware processor, a biosensor device operably connected to the mobile device, the biosensor device wearable by the user, and the at least one hardware processor performing a series of steps to manage health condition, the series of steps including monitoring for an irregularity, identifying a symptom, evaluating the symptom, and based on the symptom the system determining a context of the symptom, [symbol] based on the learned information.

The system may include the step of correlating the symptom with a context which corresponds with onset of the symptom. The system may include the steps of detecting the context in a future of the individual and intervening with the individual to prevent or mitigate the symptom.

The operations of the system may further include the steps of adapting the operations to improve the efficacy of the intervening with the individual wherein the determining the efficacy of the intervening with the individual comprises querying the individual with a dialog operation of the system.

In embodiments, to identify the irregularity as either associated with the symptom of the health condition or not associated with the symptom of the health condition, the system may prompt the individual to provide information about the irregularity. The prompt, which may take the form of an automated query, may occur as part of a dialog operation of the system, may occur as part of a dialog with a third party such as a healthcare provider, or both. A purpose of the prompt is to confirm the irregularity as being either a true positive symptom of the health condition or a false positive symptom of the health condition. Through repeated use, the AI system may adapt to variations observed in the one or more biological properties of the individual and be better able to differentiate true positive symptoms from false positive symptoms. Accordingly, in embodiments, the system may automatically evaluate the irregularity with little or no input from the individual. In embodiments, the system may associate the symptom with a context which corresponds with onset of the symptom, such as a calendar entry for an event which has been associated with the symptom in the past. In this manner, the system can identify the context in the future and intervene to prevent or mitigate the symptom before it occurs.

In another aspect, the present system and method provides a system for management of a health condition, comprising a biosensor device, optionally combined with one or more of a computational servers and a mobile device that operably connects the biosensor device to the computational server. In embodiments, the biosensor device comprises an optical data acquisition system configured to perform heart rate detection. After activation of the optical data acquisition system, the mobile device receives and processes a biosensor data stream from the biosensor device and relays a mobile data stream to the computational server for processing by a computer algorithm, such as a machine learning (ML) algorithm or an artificial intelligence (AI) algorithm, of the computational server. In embodiments, the algorithm may be configured for detection of stressors, personalization of symptom alerts, or both.

In another aspect, the present system and method provides a method for managing a health condition, includes monitoring, via a biosensor device, a biological property of an individual operably connected to the biosensor device; detecting, via a mobile device operably connected to the biosensor device, an irregularity of the biological property; and evaluating a symptom of the health condition, wherein the symptom is correlated with the irregularity. The evaluation of the symptom may consider previous symptoms experienced by the individual, previous symptoms experienced by other individuals, or both. In addition, in embodiments, the method further comprises reporting a status of the individual to a third party. The status may include information about the individual's health or mental health, and the third party may include any party who may need to know the status, including but not limited to family, friends, neighbors, relatives, healthcare professionals, law enforcement, social workers, and the like.

In embodiments, the present system and method provides early prediction of contexts, stressors, or triggers associated with particular symptoms, seamless data capture of personalized event triggers through virtualized natural language processing (NIP) interviews with the individual, and delivery of software-based psychotherapy based on an artificial intelligence (AI) recommendation engine, to reduce symptoms. In embodiments, the system and method provides real-time or just-in-time intervention approaches, as well as integration of human healthcare providers (e.g., a clinician or a therapist) to supervise progress while scaling available healthcare [symbol] In this manner, individuals may be better able to manage their health conditions with little or no traditional or in-person intervention.

In embodiments, a biosensor device (e.g., a wearable biosensor device) captures one or more of heart rate variability (HRV), electrodermal activity (EDA), temperature, Sp02, movement, and respiration, and this data may be combined with other passively collected data sources to detect negative stressors which are correlated with a symptom of a condition, such as a feeling of anxiousness which is correlated with anxiety. The system may learn to detect and even anticipate these events based on sensor data, additional data, or both. Once detected, a computer program of the system may provide one or more just-in-time (JIT) activities, such as a positive activity or another intervention to mitigate events in real-time. When these events are predictable (e.g., right before a stressful event, such as an exam or a presentation), the system and method helps the user manage a mental health condition, such as stress, anxiety, or depression, before the symptom begins. Furthermore, the system's speech interface uses AI and natural language processing (NIP) to “interview” the user, collecting subjective data unique to that user. These interviews pinpoint the stressful event and specific triggers. The results of these AI/NIP highly personalized interviews and biometrics enable short- and long-term strategy selection for a therapy such as cognitive behavioral therapy (CBT), dialectical behavior therapy (DBT), acceptance and commitment therapy (ACT), positive activities, breathing exercises, and other method-based interventions to help users reduce their mental health symptoms.

The system and method leverages several significant innovations driven by artificial intelligence (AI) engines to address the long-felt need for scaling the healthcare system, particularly regarding mental health treatment, to improve care and quality of life. These innovations include multimodal objective biometrics from wearable technology, AI for early detection and determination of intervention strategies, and the seamless integration of AI workflows with human clinician experts.

Another object of the present system and method is to provide systems, methods, and machine-readable media that may be readily employed to benefit at-risk populations and scale a strained healthcare system to benefit those most in need.

Other objects, features and advantages of the present system and method will become apparent from the following detailed description taken in conjunction with the accompanying drawings.

### BRIEF DESCRIPTION OF THE DRAWINGS

The embodiments set forth in the drawings are illustrative and exemplary in nature and not intended to limit the subject matter. The following detailed description of the illustrative embodiments can be understood when read in conjunction with the following drawings, where like structure is indicated with like reference numerals and in which:

FIG. 1 depicts an exemplary HRV graph according to one or more embodiments shown and described herein;

FIG. 2 depicts an exemplary skin conductance response (SCR) graph, showing EDA as a function of time according to one or more embodiments shown and described herein;

FIG. 3 depicts a diagram of an exemplary system, according to the present specification according to one or more embodiments shown and described herein;

FIG. 4 depicts a diagram of an exemplary biosensor device, configured for use with the system and method of the present specification according to one or more embodiments shown and described herein;

FIG. 5 depicts a flowchart of a use of a system to perform an exemplary method, according to the present specification according to one or more embodiments shown and described herein; [symbol] sources of stressors or triggers according to one or more embodiments shown and described herein;

FIG. 7 depicts a flowchart of a use of a system to perform an exemplary method for addressing anxiety or a similar mental health condition, according to the present specification according to one or more embodiments shown and described herein;

FIG. 8 depicts an exemplary user interface of a mobile device, in use to view a plurality of options available in a software application of the present specification according to one or more embodiments shown and described herein;

FIG. 9 depicts an exemplary dialog interface of the mobile device, in use to view feedback to a question prompted by the system of the present specification according to one or more embodiments shown and described herein;

FIG. 10 depicts an exemplary intervention interface of the mobile device, in use to view a plurality of interventions available to a user in need thereof according to one or more embodiments shown and described herein;

FIG. 11 depicts an exemplary intervention interface of the mobile device, in use to view a plurality of favorite activities of the user in need thereof according to one or more embodiments shown and described herein; and

FIG. 12 depicts a block diagram of a machine in the example form of a computer system within which instructions may be executed to cause the machine to perform any one or more of the methodologies discussed herein according to one or more embodiments shown and described herein.

### DETAILED DESCRIPTION

Reference is made herein to the attached drawings. Like reference numerals are used throughout the drawings to depict like or similar elements of the system and method. The figures are intended for representative purposes only and should not be considered limiting in any respect.

Because a higher percentage of young people experience psychological distress compared to older people, there exists a substantial need for an effective innovation in the mental healthcare space, particularly for these groups.

In the shown embodiment, the system is configured to reduce stress, anxiety, or depression of a user using a biosensor device and AI-based continuous monitoring. Because the user is continuously monitored, the system may be configured to collect data that pertains to a resting state as well as an aroused state. In an exemplary embodiment, the resting state may correlate with a relatively lower frequency of symptoms detected by the system, and the aroused state may be evaluated—through questioning of the user for psychological input or through analysis of the symptom data for physiological input—to determine whether the symptom is truly correlated with a particular health condition. For example, if a user becomes aroused through exercise or ordinary physical exertion, the system may ask the user whether this arousal represents a symptom of the health condition or may determine this without user input, for example, by computationally evaluating one or more parameters of the arousal. By collecting and analyzing enough information that pertains to the user, the system may learn which symptoms are detrimental and require intervention, which symptoms are mildly detrimental and may not require intervention, and which symptoms are not detrimental and do not require intervention. In this manner, the system may adapt, change, or evolve with a user to learn how to best intervene for delivery of care.

In some embodiments, the system occurs on a mobile device or user device. It is noted that the terms “mobile device” and “user device” may be used interchangeably as used and defined herein. The “mobile device” or “user device” may be any cell phone, tablet, smart phone, mobile display, other display, self-contained computer, or any other similar device whether mobile or otherwise immobile [symbol] other being (living or not).

In embodiments, the systema and method enables the individual operably connected to the biosensor device to experience biofeedback, optionally as part of a therapeutic intervention. For example, the individual can be made consciously aware of their own heart rate (HR), electrodermal activity (EDA), respiration rate, or a combination thereof, based on feedback from the biosensor device. One or more of these exemplary biological properties may be displayed on a computer screen, such as a screen of the individual's smartphone, and as the individual undergoes a therapeutic intervention, they may be able to observe improvement of one or more of these exemplary biological properties as the symptom is managed. For example, the HR, the EDA, or the respiration rate may decrease with a controlled breathing exercise or a CBT intervention, and the individual then knows they are doing better, and they feel better as well. In embodiments, biofeedback may be presented to the individual before, during, after, or independent of a therapeutic intervention. If the biofeedback is presented to the individual just as the individual begins experiencing a symptom, this may enable the individual to self-correct, alone or in combination with the therapeutic intervention. In this manner, the individual may not necessarily need a full therapeutic intervention to cope with the symptoms and may be able to self-soothe or self-correct with biofeedback provided by the system.

The system may be configured to reduce stress, anxiety, or depression with a biosensor device and AI-based continuous monitoring for signs of symptoms. The symptom detection and management are accomplished by monitoring one or more of heart rate variability (HRV), electrodermal activity (EDA), temperature, Sp02, movement, and respiration, the use of ML and signal processing, and AI-based just-in-time symptom management activities. In this manner, the system can intervene in real-time within an individual's everyday life, in a microenvironment that is inaccessible to traditional treatment methods, to increase prevalence, effectiveness, and accessibility of care, where “real-time” is defined as collection and providing data/information and processing within milliseconds so that it is available virtually immediately as feedback In other words, the system processes information and data as it is received so that the user receives the result of the processed information and data quickly after data is collected. It is particularly advantageous to provide feedback and/or interventions in real-time so that a user can mitigate their stressful or anxiety inducing situation quickly thereby ideally leading to improved mental health. Real-time computing is a common term for hardware and software systems subject to a “real-time constraint”, for example from event to system response. Real-time programs must guarantee response within specified time constraints.

In embodiments, the system may include a camera system (which may be configured to capture still images, video, or both), configured to image an individual, process one or more images of the individual, and produce one or more biosignals from the one or more images of the individual which may be processed by the system. Exemplary biosignals which may be produced by the camera system include one or more of heart rate variability (HRV), electrodermal activity (EDA), temperature, Sp02, movement, and respiration. In an exemplary embodiment, the camera system may be comprised of a camera-based vital signs detector (such as Philips® VitalSigns®) and may be configured for contactless heart and breathing rate monitoring. In embodiments, the camera system may be configured to detect over 97% of the respiration cycle of a particular subject or individual by utilizing remote photoplethysmography (PPG) to track changes in the chest and abdomen to determine respiration rate as well as changes in skin color caused by heart rate. In embodiments, the camera system may be robust to operate even in the presence of motion, different light levels, and the like. As such, any device which may be a source of biosignals may be utilized in the present system and method, according to need.

The system of the present system may include both a biosensor device (e.g., a wrist, finder, head, chest strap, or any other body part worn wearable device by means of a sticker, strap, wing, jewelry . . . etc.) and a software application deployed on a user's mobile device. The biosensor device may be worn by the user or may be auxiliary (e.g. wall mounted camera, external camera or infrared reader, located on the mobile device, or any other sensor used to measure and collect biometric data). The system provides an end-to-end system [symbol] heart rate variability and electrodermal activity, as well as other passively collected data sources as biometric inputs to detect negative stressors which are correlated with stress, anxiety, or depression. Artificial intelligence (AI) algorithms of the system are trained and validated to anticipate negative stressor events based on sensor data. Using AI algorithms enables intelligence demonstrated by machines, as opposed to natural intelligence displayed by animals including humans which can be performed faster as compared to natural intelligence. Once detected, the software application provides a combination of just-in-time (or real-time) activities including pre-selected positive activities and interventions to mitigate events in real-time to reduce stress, anxiety, or depression before it begins. Furthermore, the software application speech interface uses AI and natural language processing (NIP) to “interview” the user, collecting subjective data unique to that user. These interviews pinpoint the stressful event and specific triggers. Results of the AI/NIP highly personalized interviews and biometrics inform short-term and long-term strategy selection for a therapy such as cognitive behavioral therapy (CBT), dialectical behavior therapy (DBT), acceptance and commitment therapy (ACT), a positive activity, a breathing exercise, general therapy, and related software application-based interventions to help users better manage symptoms over time where “general therapy” is any other therapy suitable for reducing a stressful or high anxiety event.

In embodiments, HRV assessment based on non-linear methods detect and measure peculiar aspects of cardiac rhythm dynamics that are not taken into account by linear techniques. Due to the complex and non-linear interactions between a number of different physiological systems that participate in HRV genesis, the non-linear methods represent promising tools for HRV assessment. Various algorithms are used to develop automated recognition systems to apply HRV measurement in clinical and daily-life situations.

In addition, in some embodiments the system includes a neural network configured to detect and classify anxiety, stress, or depression, with a high degree of accuracy. In embodiments, the system utilizes a rule-based algorithm for health condition detection based on one or more of heart rate variability (HRV), electrodermal activity (EDA), temperature, Sp02, movement, and respiration using a MI algorithm. In some embodiments, a Tree-based Pipeline Optimization Tool (TPOT) may be utilized, which is an automated machine learning tool that optimizes machine learning pipelines using genetic programming, to build the best model. By using this mixed-methods approach, the shortcomings associated with individual methods have been overcome. In embodiments, the system may accurately detect when the user is in one of three states, including calming task, negative stressor task, and positive stressor task.

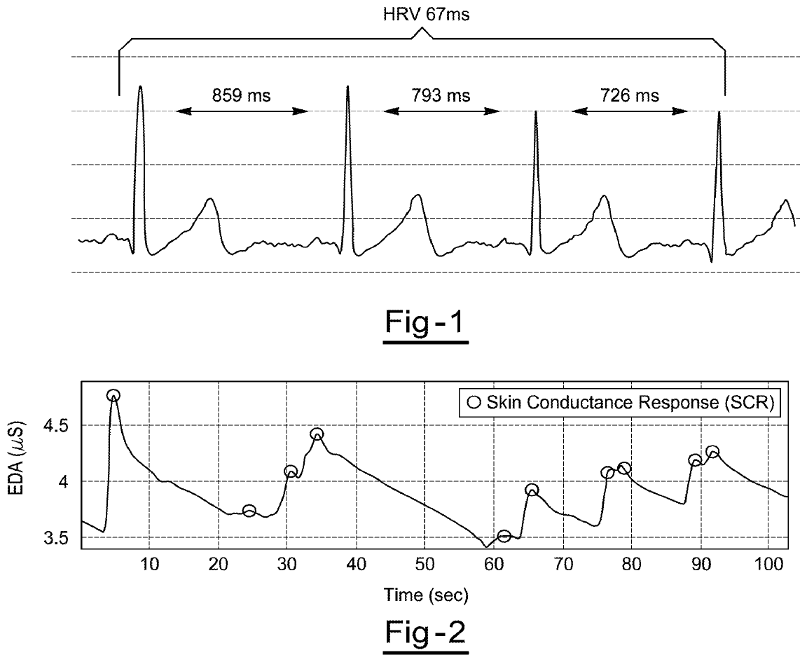

Referring now to FIG. 1, there is depicted an exemplary HRV graph. Fluctuations in heart rate result from complex, non-linear interactions between a plurality of different physiological systems, but the autonomic nervous system (ANS) is most prominent and HRV is a function of the heart-brain interaction. The parasympathetic and sympathetic nervous systems regulate the body at rest and during fight-or-flight scenarios, respectively, and significantly impact HRV, making HRV a reflection of a person's physiological and psychological wellbeing. Specifically, HRV is a reliable parameter for analyzing the chronic shift toward increased sympathetic and decreased parasympathetic activity typical of autonomic imbalance. This imbalance has largely been associated with chronic stress and emotional exhaustion, as well as anxiety disorders, depression, and other psychological disorders. In another aspect, HRV is an indicator of better general health status, self-regulatory capacity, adaptability, and emotional and stress resilience. Specifically, low HRV is an indicator of abnormal and insufficient adaptation of the ANS and may be associated with harmful health events, especially when sustained for a prolonged period.

In embodiments, the present system and method uses HRV as a neurocardiac function biomarker to detect and classify mental stress, anxiety disorders, and depression. The biosensor device measures HRV (e.g., a wearable device placed on a wrist of the individual, on a torso of the individual, on an ear of the individual; a video recording of a portion of the individual's body, such as the individual's face) through photoplethysmography (PPG) using the acquired signal to feed into software to calculate several parameters that are time-based, [symbol] the camera. In embodiments, HRV may be measured over different time periods, from less than 5 minutes to 24 hours, which is useful for clinical HRV assessments.

Referring now to FIG. 2, there is depicted an exemplary skin conductance response (SCR) graph, showing EDA as a function of time produced by an exemplary biosensor device worn on a wrist, hand, finger, or any other part of the body. Sweat gland activity influences skin conductance and thermoregulation. Sweat glands are regulated by the ANS as well and are modulated during the fight-or-flight response. In particular, the sympathetic branch stimulates sweat glands and elevates sweating. Since the number of active sweat glands increases with sympathetic activation, skin conductivity is proportional to sweat secretion; a change in skin conductance at the surface reflects the sympathetic activity and provides a non-invasive and sensitive measure of EDA.

During chronic stress, the ANS is out of balance, and a lack of equilibrium exists between the sympathetic and parasympathetic systems. Along with HRV, EDA is one of the most important physiological signals for detecting stress. The most common markers associated with chronic stress are elevated levels of EDA. Chronic stress hyperactivates the sympathetic branch and disrupts the autonomic balance. Because EDA is solely determined by the sympathetic activity, which is predominant in stress states, EDA may be a suitable measure of ANS activity induced by stress. EDA measurement may provide the emotional state of an individual in real time, without any verbalization. Phasic features can be helpful in evaluating stress, anxiety, and depression. In research settings, EDA has quantified attention, memory, decision-making, emotion, and has acted as a predictor of normal and abnormal behavior and other psychological constructs. EDA, as part of a technology that automatically recognizes stress, anxiety, and depression, is a powerful tool both in clinical settings and in daily life.

EDA was traditionally measured by sensors placed on high-density sweat gland areas (e.g., fingers, palms, etc.). The system of the present system allows EDA signal collection outside of the laboratory setting with a wearable device that can continuously measure EDA with wearable designs that integrate sensor contacts. EDA can be assessed by measuring the electrical conductance, resistance, impedance, or admittance of the skin via endosomatic and exosomatic methods to distinguish the tonic and phasic components of the electrical signal. EDA's tonic component, skin conductance level (SCL), is related to the slowly varying skin conductance level, and corresponds to baseline level of skin conductance. SCL is computed as a mean of several measurements taken during a specific non-stimulation rest period. Thus, SCL is slowly changing and measures general psychophysiological activation (that can vary substantially among individuals). The phasic component is the rapidly fluctuating part of EDA that corresponds to the response to a specific and discrete stimulus, such as when the sudomotor nerve is activated. Generally, the increase in skin conductance starts 1 to 4 seconds after stimulus exposure, and persists for 1 to 3 seconds, allowing different amplitudes to be easily measured. Difficulties arise when responses occur continually and close together in time, making it difficult to distinguish between individual peak events. Algorithms have been developed to extract overlapping peaks and filter noise artifacts. In embodiments, the present system measures raw conductance and then applies an algorithm to extract the tonic and phasic components into separate features, for example, Phasic Peaks, Max and Variance, Tonic Peaks, Mean and Variance, and the like. In this manner, the peak events can be characterized, and nervous system activity monitored and quantified.

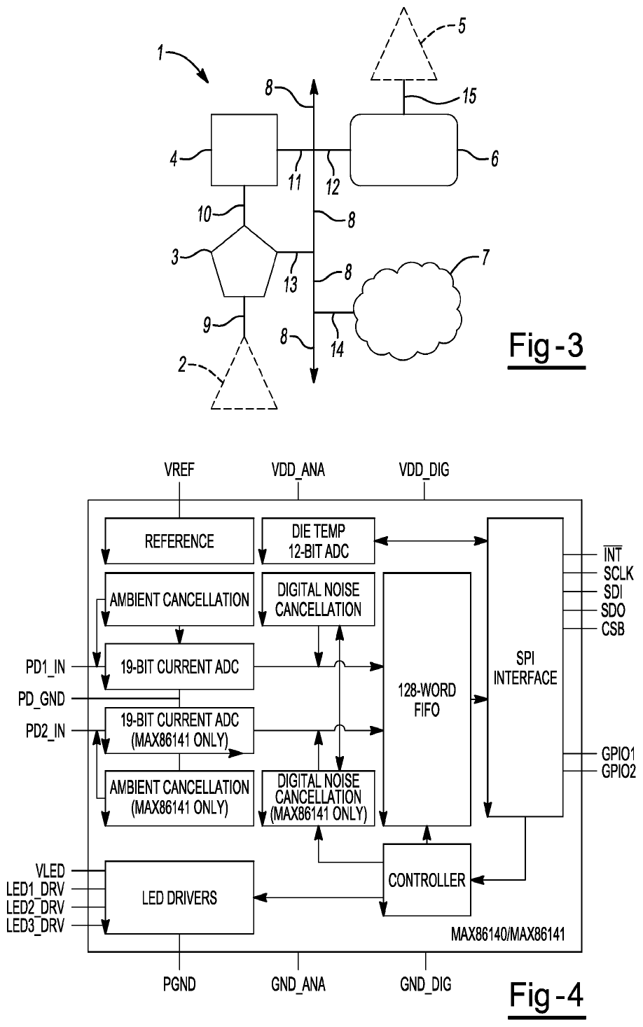

Referring now to FIG. 3, there is depicted a diagram of an exemplary system, according to the present system. Generally, the present invention provides a system 1 for management of a health condition, comprising one or more devices (e.g., 3, 4, 6, 7) configured to monitor a biological property of an individual 2, detect an irregularity of the biological property, and evaluate a symptom of the health condition. In the shown embodiment, the individual 2 is optionally connected with a healthcare professional 5 or another individual capable of aiding the individual 2. In this manner, the individual 2 may communicate with the healthcare professional 5 if desired or needed for real-time or expedited delivery of care to the individual 2. [symbol] machine-readable media storing instructions that, when executed by the one or more hardware processors, cause the one or more hardware processors to perform operations for management of a health condition. The operations which may be performed by the one or more hardware processors comprise monitoring a biological property of an individual 2, identifying an irregularity of the biological property as a symptom of the health condition, and intervening with the individual 2 to alleviate the symptom.

In some embodiments, the one or more hardware processors and the one or more machine-readable media may be components of a biosensor device 3 of the system. In embodiments, the biosensor device 3 may be configured to perform operations of the system 1. In this manner, the biosensor device 3 may not need to communicate with another device, such as a mobile device 4 or a networked computational server 7, to perform the operations. In embodiments, the biosensor device 3 may be the only device of the system 1, and in this manner, the system 1 may not comprise the mobile device 4, the networked computational server 7, or the healthcare console 6. In this manner, the operations of the system 1 may be performed by the biosensor device 3 alone.

In some embodiments, the one or more hardware processors and the one or more machine-readable media may be components of a mobile device 4 of the system 1, and the system 1 may comprise a biosensor device 3 operably connected to the mobile device 4. In some embodiments, the biosensor device 3 may be utilized as a source of biosensor data which is sent to the mobile device 4, e.g., via connection 10, for the operations of the system 1 to be performed by the mobile device 4. In embodiments, the biosensor device 3 and the mobile device 4 may be the only devices of the system 1, and in this manner, the system 1 may not comprise the networked computational server 7 or the healthcare console 6. In this manner, the operations of the system 1 may be performed by the biosensor device 3 and the mobile device 4 alone.

In some embodiments, the one or more hardware processors and the one or more machine-readable media may be components of a networked computational server 7 of the system 1, and the system 1 may comprise a biosensor device 3 operably connected to the networked computational server 7. In embodiments, the biosensor device 3 may be utilized as a source of biosensor data which is sent to the networked computational server 7, e.g., via connections 13, 8, and 14, for the operations of the system 1 to be performed by the networked computational server 7, and in this manner, the system 1 may not comprise the mobile device 4 or the healthcare console 6. In this manner, the operations of the system 1 may be performed by the biosensor device 3 and the networked computational server 7 alone. However, in embodiments, the system comprises the mobile device 4 that operably connects the networked computational server 7 and the biosensor device 3, e.g., through connections 10, 11, 8, and 14, for the operations of the system 1 to be performed by the networked computational server 7, and in this manner, the system 1 may not comprise the healthcare console 6. In this manner, the operations of the system 1 may be performed by the biosensor device 3 and the networked computational server 7, with the mobile device 4 as well.

Connections 8, 9, 10, 11, 12, 13, 14, and 15 may include, but are not necessarily limited to, any known wired or wireless connection type deemed suitable for transmission of data, such as digital data. Generally, these connections may be necessary to enable components of the system (e.g., a biosensor device 3, a mobile device 4, a healthcare console 6, and a computational server 7) to intercommunicate. Exemplary connection types that may be suitable for one or more of connections 8, 9, 10, 11, 12, 13, 14, and 15 include ethernet or other wired connection type, Wi-Fi® connectivity, Bluetooth® connectivity, or other electromagnetic or radio wave connectivity as needed according to a particular embodiment. It is contemplated that these connections be unidirectional or bidirectional in nature, but in particular, may be bidirectional to facilitate inter-device communication.

In the shown embodiment, the individual 2 is operably connected to a biosensor device 3 by connection 9. Connection 9 may be representative of a physical connection, as may be utilized with attachment of a biosensor device to the individual 2, but particularly, connection 9 may be representative of an operable connection configured to enable observation of the biological property of the individual [symbol] sensor for EDA measurement, an optical connection for performing pulse oximetry or heart rate detection for HRV measurement, or a combination thereof. In like manner, and consistent with embodiments of the connection 9, the biosensor device 3 may be configured to read one or more biological properties, including but not necessarily limited to a heart rate (HR), a heart rate variability (HRV), an oxygen saturation (Sp02), an electrodermal activity (EDA), a breathing rate (BR) or respiration, movement (e.g., via an accelerometer), a body temperature (BT), a blood sugar level, a perspiration level, or a body metric, and any combination thereof. The body metric may be any data regarding a user's measurable biological properties not specifically enumerate herein or not yet contemplated capable of measuring any measurable data about a biological property of a user or other being. In embodiments, the biosensor device 3 comprises an optical data acquisition system that is configured to perform pulse oximetry (for Sp02 determination) and heart rate detection (for HR or HRV determination). In embodiments, the biosensor device 3 may be configured for placement on a wrist, a finger, or an ear of the individual. In this manner, the blood oxygen level may be optically determined, along with other parameters accessible from these body parts, according to need.

In the shown embodiment, the one or more devices comprises a mobile device 4 and the biosensor device 3, wherein the mobile device 4 is operably connected to the biosensor device 3 by operable connection 10. Operable connection 10 may include any connection suitable for electronic data transmission, for example, a wired connection, one or more wireless data connections, or a combination thereof. In embodiments, the biosensor device 3 is operably connected to the mobile device 4 by the operable connection 10, wherein the operable connection 10 is comprised of a Bluetooth® wireless connection. In this manner, the biosensor device 3 is maximally compatible with existing mobile devices.

In the shown embodiment, the mobile device 4 is connected to a network 8, such as the world-wide web or internet, by an operable connection 11. Operable connection 11 may include any connection type suitable for data transmission, including a wired connection or a wireless connection. Operable connection 11 may be direct or indirect, and may proceed through any number of routers, cell towers, satellites, and the like. In this manner, the present system and method may be configured for use with any of a variety of different network designs or topologies, according to need and availability of network infrastructure in an embodiment. As ordinarily understood, network 8 may include but is not necessarily limited to a trans-continental network such as the world-wide web or internet. Network 8 may include any number of connection points, as would be understood by one having ordinary skill in the art.

In the shown embodiment, the biosensor device 3 is operably connected to the network 8 by operable connection 13. In embodiments, the biosensor device 3 may contain one or more networking functionalities, such as one or more wireless transceivers, to enable this functionality. In embodiments, the operable connection 10, the mobile device 4, and the operable connection 11 may not be needed for full functionality of the system 1. For example, in embodiments, the biosensor may include a non-transitory computer-readable medium containing data storage and computer-executable instructions thereon which effectively replace or supplant the need for the mobile device 4. In this manner, the number of components of the system 1 may be reduced or maintained at a level manageable for a particular need, such as a patient that may not utilize the mobile device 4 to use the system.

In the shown embodiment, the system comprises the computational server 7, wherein the computational server 7 comprises a networked server, operably connected to the mobile device 4 and configured to execute a machine learning (ML) algorithm to evaluate the severity of the symptom of the health condition. The computational server 7 is operably connected to the mobile device 4 by the network 8, and by network connection 14. Generally, network connection 14 may be any wired or wireless connection suitable for data transfer, and in this manner, the computational server 7 may be connected to the network 8 as needed. [symbol] console 6, the computational server 7, or a combination thereof, may include a module for event-driven activities, a module for natural language understanding (NLU), a module for speech to text (ASR), or a combination thereof. The NLU may be utilized as a source of information that may be used in a diagnosis or in an improving of an intervention. These modules may be available as executable computer algorithms, in the form of computer-readable instructions stored in a non-transitory computer-readable medium of one or more devices such as the biosensor device 3, the mobile device 4, the healthcare console 6, the computational server 7, or a combination thereof. In this manner, as the system 1 operates, biosensor data may be processed, and responses produced accordingly. Generally, the computational server may include any suitable server or cloud-based networking infrastructure or equivalent combination of computational hardware which effectively enables cloud-based computing and communication.

In some embodiments, one or more of the one or more devices (3, 4, 6, 7) comprises a history of the individual 2, wherein the history comprises data that pertains to a previous symptom, a previous intervention, an efficacy of a previous intervention, or a combination thereof. In this manner, the system 1 may learn from previous interactions with the individual 2 to anticipate symptoms and improve interventions. In embodiments, the computational server 7 may include a data structure for storage and retrieval of therapy lessons, and a data structure for storage and retrieval of user data, sensor data, audio files, chat logs, or a combination thereof. In this manner, the history of the individual may be stored on the computational server.

In some embodiments, the healthcare console 6 may be comprised of any suitable networked computing device, such as a mobile device (i.e., mobile phone, tablet, etc.), or a personal computer or workstation. The healthcare console 6 may be configured to enable the healthcare professional 5, such as a doctor, a psychiatrist, a therapist, and the like, to chat with the individual 2 and provide therapy recommendations based on a current situation in view of the history of the individual 2. The healthcare console 6 is connected to the network 8 by connection 12, which may be a wired or a wireless data transfer connection.

Referring now to FIG. 4, there is depicted a diagram of an exemplary biosensor device, configured for use with the present system and method. In the shown embodiment of FIG. 4, the biosensor device is an ultra-low power, completely integrated, optical data acquisition system. The biosensor includes three programmable high-current light-emitting diode (LED) drivers configurable to drive up to six LEDs and one or two optical readout channels that can operate simultaneously when present in a pair. The biosensor includes a low-noise signal conditioning analog front-end (AFE) including 19-bit ADC, an industry-leading ambient light cancellation (ALC) circuit, and a picket fence detect and replace algorithm. The biosensor device is ideal for a wide variety of optical-sensing applications, such as pulse oximetry or heart rate detection. In embodiments, the biosensor device is configured for use to detect the individual's heart rate.

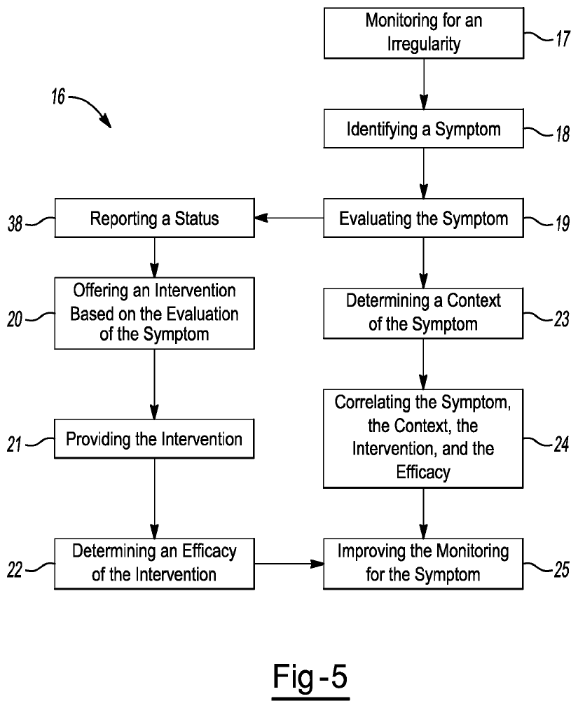

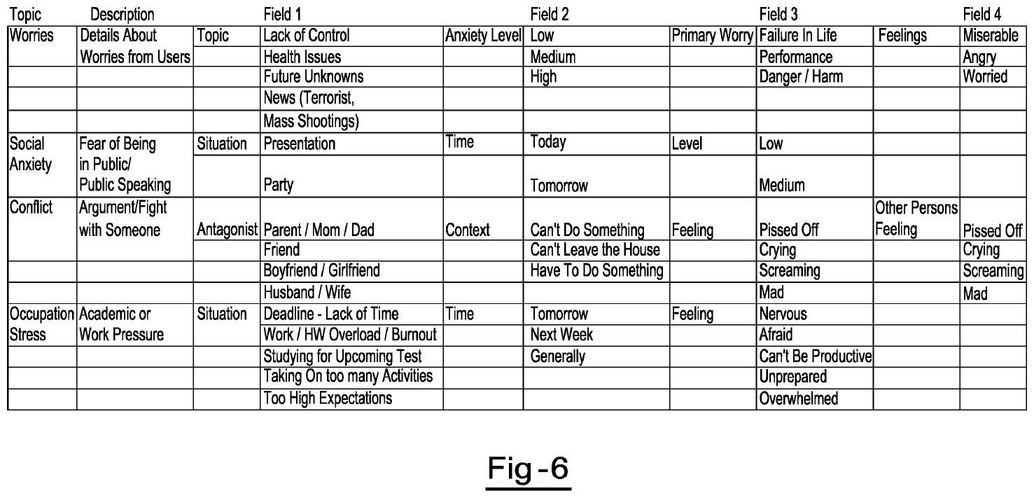

Referring now to FIG. 5, there is depicted a flowchart of a use of a system to perform an exemplary method 16. In the shown embodiment, the method 16 comprises monitoring 17, via a biosensor device, a biological property of an individual operably connected to the biosensor device; identifying a symptom 18, via one or more devices of the system, as an irregularity of the biological property; and evaluating 19 the symptom of the health condition, wherein the symptom is characteristic of the health condition.

In embodiments, the method further comprises reporting 38 a status of the individual to a third party. The status may include information about the individual's health or mental health, and the third party may include any party who may need to know the status, including but not limited to family, friends, neighbors, relatives, healthcare professionals, law enforcement, social workers, and the like. In embodiments, the third party may include one or more persons physically near the individual. In embodiments, the reporting the status 38 of the individual may occur through electronic communication over a telephone network, a computer network, or both. In embodiments, the reporting the status 38 of the individual may occur via a proximity notification such as an audible alert, a visual alert, or an announcement that draws the attention of passersby who may be able to assist the individual. [symbol] symptom; and intervening 21 with the individual to initiate an activity intended to alleviate the symptom. After the intervening 21, the method 16 may include determining 22 an efficacy of the intervention by querying the individual (e.g., via a dialog function of the system), by additionally monitoring the biological property via the biosensor device, or a combination thereof. In embodiments the mobile device detects a change in the irregularity of the biological property, such that the change is associated with an improvement in the symptom. In such a scenario, an efficacy of the intervention may be determined to be positive.

The providing 21 the intervention step of the method may include a series of therapeutics (e.g., mindfulness, meditation, etc.) in the form of video, audio, recommendations, activities, or exercises or one or more therapies in the software application for overcoming common situations. Exemplary exercises or therapies include cognitive behavioral therapy (CBT), dialectical behavior therapy (DBT), acceptance and commitment therapy (ACT), a positive activity, a breathing exercise, and any combination thereof. In embodiments, a set of DBT-based responses for frequently occurring situations are encoded to be used during, after, and eventually, before the occurrence of the stressful event. Responses may be as simple as breathing exercises, going for a walk, etc. Users also have access to a therapist to provide support for other situations that may not be handled by the software application, and a therapist may be made available via the system if the user prefers to communicate with a live person. The software application incorporates one or more biosensor devices for real-time detection, symptom reduction techniques, and therapies for both immediate and long-term care. By integrating these components and technologies, the system provides elements of therapy normally used in high-intensity interventions, like changing core beliefs through pattern-recognition in a person's daily life, where intervention matters most.

In the shown embodiment, the method 16 further comprises determining 23 a context of the symptom. This determination may be accomplished by prompting the individual or user with one or more questions or dialog functions to help the system understand how the symptom occurred. After determining 23 the context of the symptom, the system may produce correlations that enable improvement of the monitoring, the intervening, or both. The system may correlate 24 the symptom, the context, the intervention, and the efficacy to understand situations in which the individual experiences negative symptoms, and situations in which negative symptoms respond to particular forms of intervention. This information can be used as part of a procedure for improving 25 the monitoring for the symptom, and the method 16 overall, as the system may be capable of adapting to the individual's needs. In this manner, the operations of the system may further comprise detecting the context in a future of the individual and intervening with the individual to prevent or mitigate the symptom. In this manner, the individual may avoid the symptom altogether using the system as disclosed herein.

Referring now to FIG. 6, there is depicted a table containing a plurality of exemplary frameworks that may be utilized by the system to understand underlying sources of stressors or triggers. These frameworks may be used, for example, as dialog functions performed by one or more devices of the system. In the shown embodiment, the frameworks include a worry framework, a social anxiety framework, a conflict framework, and an occupational stress framework. Each framework includes a description and a plurality of fields that correspond to aspects of that framework. During use of the framework by the individual and user of the system, one or more frameworks may be utilized during a particular dialog function.

In embodiments, the software application aggregates all data sources and provides user workflows and feedback, and integrates an Interview Interface to ensure simple, timely, and rich data collection to drive context awareness to detection algorithms. The Interview Interface functions by first creating frames that capture the specific mindset issue that the user is facing. Using a natural language processing technology such as Google's Bidirectional Encoder Representations from Transformer (BERT) framework, the software application collects data from the user on the exact nature of their issue(s). Once information is collected, the software application uses a dialog to walk the user through a series of steps to assess the source of their stress. For example, in the “Worries Framework,” the software [symbol] times of the day or week that can be related to specific types of stressors. FIG. 6 includes examples of several kinds of frames that may be utilized in a system and method, to leverage this data. These frames may be used for creating histories of the person's issues for therapy and improved interactive dialogs. They may also be used for identifying common cognitive distortions (filtering, polarized thinking, “should”, blaming, etc.) and to guide the user through them.

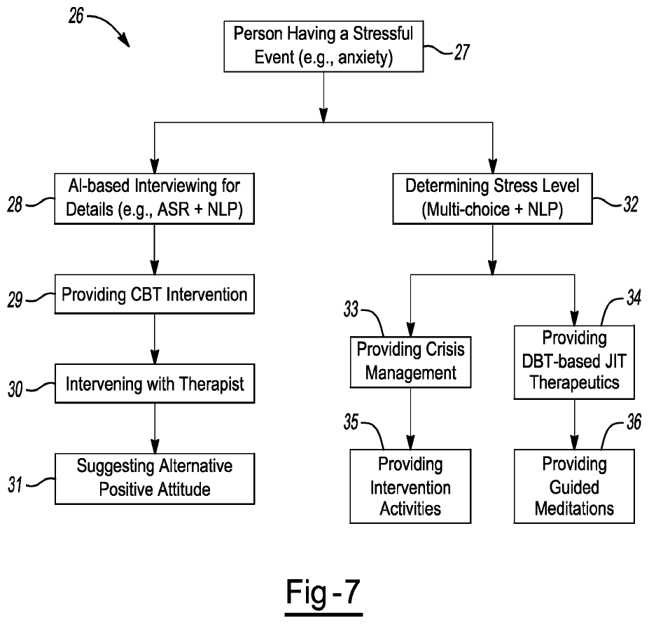

Referring now to FIG. 7, there is depicted a flowchart of a use of a system to perform an exemplary method for addressing anxiety or another mental health condition, according to the present specification. In the shown embodiment of FIG. 7, a method 26 for managing stress is presented. The method 26 comprises a person having a stressful event 27, after which the system sets out determining 32 the stress level (e.g., through a multiple-choice or natural language processing (NIP)) immediately thereafter and performing 28 an AI-based interview for additional details later on (e.g., through speech to text (ASR) and NIP). After determining 32 the stress level, the method 26 may comprise providing 33 crisis management or providing 34 a therapeutic such as a DBT-based just-in-time (JIT) therapeutic, depending on need. Exemplary therapeutics include guided meditation and box breathing, and selection of a particular therapy or intervention may be based on relative severity of the symptom. For example, if the stress is severe, providing 33 crisis management may be preferred, and if the stress is mild, providing 34 DBT-based JIT therapeutics may be preferred. After providing 33 crisis management, for example, after the individual has calmed down, the method 26 may include providing 35 one or more intervention activities to the individual to further reduce stress. In embodiments, after providing 34 DBT-based JIT therapeutics, the method 26 may include providing 36 guided meditations to the individual. If after providing 28 the AI-based interview it is determined that a CBT analysis is needed, the method 26 may include providing 29 a CBT analysis to the individual, optionally followed with intervening 30 via a therapist, or suggesting 31 an alternative positive attitude. Because the method 26 follows a logical and structured decision-making framework, the individual is guided in an intuitive manner to much-needed care before, during, and after the stressful event.

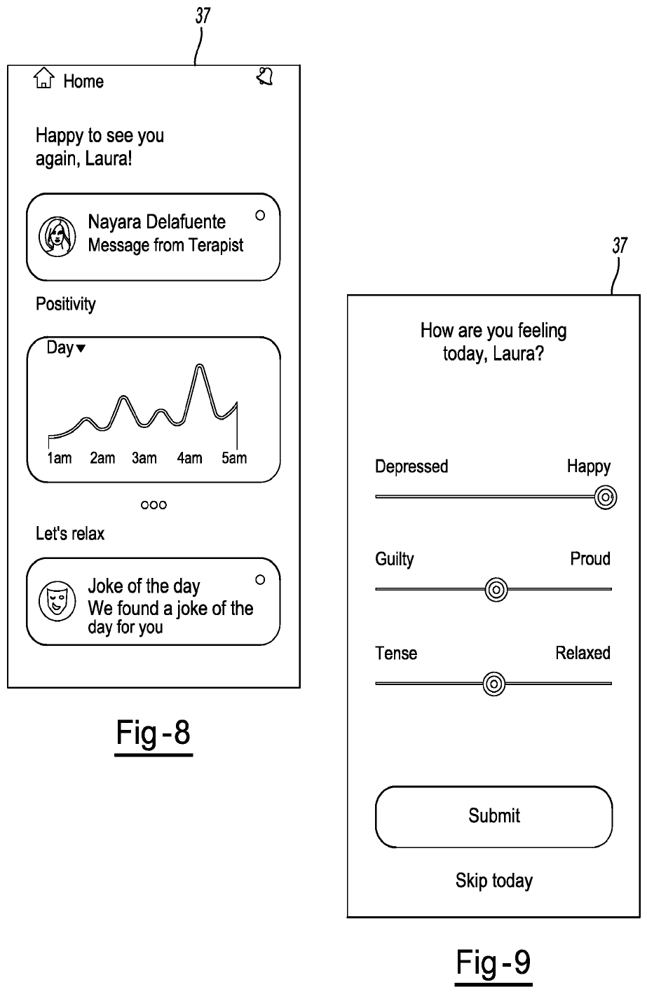

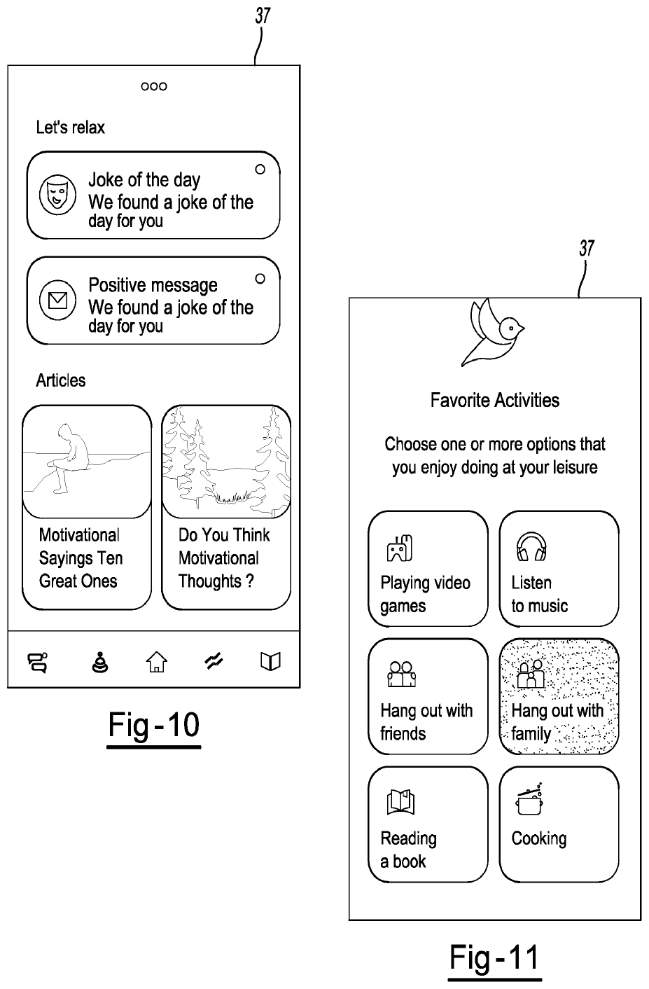

Referring now to FIGS. 8, 9, 10, and 11, there are depicted exemplary user interfaces 37 of a mobile device of the system, in use to view a plurality of options available in a software application (FIG. 8), view feedback to a question prompted by the system (FIG. 9), view a plurality of interventions available to a user in need thereof (FIG. 10), and view a plurality of favorite activities of the user in need thereof (FIG. 11). In embodiments, the activity for a particular intervention by the system and method may include one or more activities selected from a group including, but not necessarily limited to, cognitive behavioral therapy (CBT), dialectical behavior therapy (DBT), acceptance and commitment therapy (ACT), a positive activity, a breathing exercise, and any combination thereof. However, in embodiments, the activity for a particular intervention may be any exercise, any therapy, or any other intervention. Additional exemplary activities include engaging in humor, receiving motivational messages, playing a game, listening to music, socializing, reading a book, cooking, and any combination thereof. Users are unique with respect to whether and how they respond to an activity, and this list may be expanded and may vary as needed.

The operations, algorithms, and methods disclosed herein may generally be implemented in suitable combinations of software, hardware, firmware, or a combination thereof, and the provided functionality may be grouped into a number of components, modules, or mechanisms. Modules can constitute either software modules (e.g., code embodied on a non-transitory machine-readable medium) or hardware-implemented modules. A hardware-implemented module is a tangible unit capable of performing certain operations and can be configured or arranged in a certain manner. In example embodiments, one or more computer systems (e.g., a standalone, client, or server computer system) or one or more processors can be configured by software (e.g., an application or application portion) as a hardware-implemented module that operates to perform certain operations as described herein. [symbol] implemented module can comprise dedicated circuitry or logic that is permanently configured (e.g., as a special-purpose processor, such as a field programmable gate array (FPGA) or an application-specific integrated circuit (ASIC)) to perform certain operations. A hardware-implemented module can also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) can be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner, to perform certain operations described herein, or both. Considering embodiments in which hardware-implemented modules are temporarily configured (e.g., programmed), each of the hardware-implemented modules need not be configured or instantiated at any one instance in time. For example, where the hardware-implemented modules include a general-purpose processor configured using software, the general-purpose processor can be configured as respective different hardware-implemented modules at different times. Software can accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules can be regarded as being communicatively coupled. Where multiple such hardware-implemented modules exist contemporaneously, communications can be achieved through signal transmission (e.g., over appropriate circuits and buses that connect the hardware-implemented modules). In embodiments in which multiple hardware-implemented modules are configured or instantiated at different times, communications between such hardware-implemented modules can be achieved, for example, through the storage and retrieval of information in memory structures to which the multiple hardware-implemented modules have access. For example, one hardware-implemented module can perform an operation and store the output of that operation in a memory device to which it is communicatively coupled. A further hardware-implemented module can then, at a later time, access the memory device to retrieve and process the stored output. Hardware-implemented modules can also initiate communications with input or output devices, and can operate on a resource (e.g., a collection of information).

The various operations of example methods described herein can be performed, at least partially, by one or more processors that are temporarily configured (e.g., by software) or permanently configured to perform the relevant operations. Whether temporarily or permanently configured, such processors can constitute processor-implemented modules that operate to perform one or more operations or functions. The modules referred to herein can, in some example embodiments, comprise processor-implemented modules.

Similarly, the methods described herein can be at least partially processor implemented. For example, at least some of the operations of a method can be performed by one of processors or processor-implemented modules. The performance of certain of the operations can be distributed among the one or more processors, not only residing within a single machine, but deployed across a number of machines. In embodiments, the processor or processors can be located in a single location (e.g., within an office environment, or a server farm), while in other embodiments the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), these operations being accessible via a network (e.g., the Internet) and via one or more appropriate interfaces (e.g., application program interfaces (APIs)). [symbol] combinations thereof. Example embodiments can be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of description language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations can be performed by one or more programmable processors executing a computer program to perform functions by operating on input data and generating output. Method operations can also be performed by, and apparatus of example embodiments can be implemented as, special purpose logic circuitry, e.g., an FPGA or an ASIC.

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine) and software architectures that can be deployed, in various example embodiments.

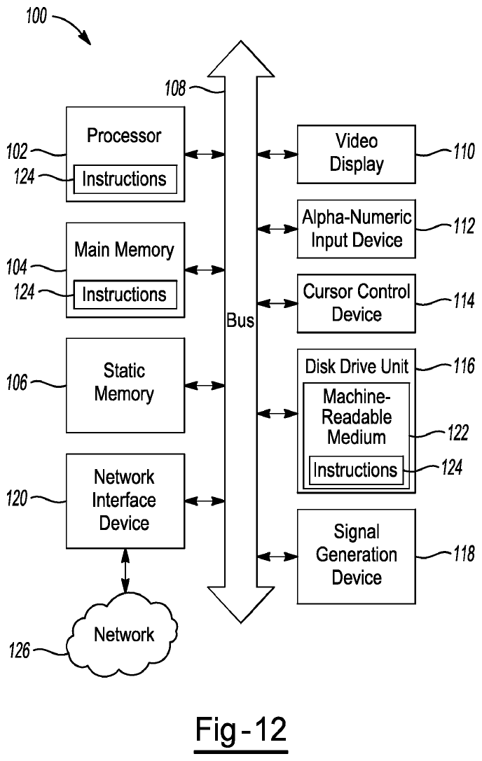

FIG. 12 is a block diagram of a machine in the example form of a computer system 100 within which instructions 124 may be executed to cause the machine to perform any one or more of the methodologies discussed herein. In alternative embodiments, the machine operates as a standalone device or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 100 includes a processor 102 (e.g., a central processing unit (CPU), a graphics processing unit (GPU), or both), a main memory 104, and a static memory 106, which communicate with each other via a bus 108. The computer system 100 can further include a video display 110 (e.g., a liquid crystal display (LCD) or a cathode ray tube (CRT)). The computer system 100 also includes an alpha-numeric input device 112 (e.g., a keyboard or a touch-sensitive display screen), a user interface (UI) navigation (or cursor control) device 114 (e.g., a mouse), a disk drive unit 116, a signal generation device 118 (e.g., a speaker), and a network interface device 120.

The disk drive unit 116 includes a machine-readable medium 122 on which are stored one or more sets of data structures and instructions 124 (e.g., software) embodying or utilized by any one or more of the methodologies or functions described herein. The instructions 124 can also reside, completely or at least partially, within the main memory 104 or within the processor 102, or both, during execution thereof by the computer system 100, with the main memory 104 and the processor 102 also constituting machine-readable media. [symbol] medium” can include a single medium or multiple media (e.g., a centralized or distributed database, and/or associated caches and servers) that store the one or more instructions 124 or data structures. The term “machine-readable medium” shall also be taken to include any tangible medium that is capable of storing, encoding, or carrying instructions 124 for execution by the machine and that cause the machine to perform any one or more of the methodologies of the present disclosure, or that is capable of storing, encoding, or carrying data structures utilized by or associated with such instructions 124. The term “machine-readable medium” shall accordingly be taken to include, but not be limited to, solid-state memories, and optical and magnetic media. Specific examples of machine-readable media 122 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 124 can be transmitted or received over a communication network 126 using a transmission medium. The instructions 124 can be transmitted using the network interface device 120 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi® and WiMAX networks). The term “transmission medium” shall be taken to include any intangible medium that is capable of storing, encoding, or carrying instructions 124 for execution by the machine, and includes digital or analog communications signals or other intangible media to facilitate communication of such software.

The foregoing descriptions of specific embodiments have been presented for purposes of illustration and description. They are not intended to be exhaustive or to limit the present appended claims to the precise forms disclosed, and modifications and variations are possible in view of the above teaching. The exemplary embodiment was chosen and described to best explain the principles of the present invention and its practical application, to thereby enable others skilled in the art to best utilize the present system and method and its embodiments with modifications as suited to the use contemplated.

With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention. It is noted that the terms “substantially” and “about” may be utilized herein to represent the inherent degree of uncertainty that may be attributed to any quantitative comparison, value, measurement, or other representation. Any values that may be modified by such terminology are also part of the teachings herein. For example, if a teaching recited “about 10,” the skilled person should recognize that the value of 10 is also contemplated.

These terms are also utilized herein to represent the degree by which a quantitative representation may vary from a stated reference without resulting in a change in the basic function of the subject matter at issue.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be [symbol] numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the Invention of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

While particular embodiments have been illustrated and described herein, it should be understood that various other changes and modifications may be made without departing from the spirit and scope of the claimed subject matter.

Moreover, although various aspects of the claimed subject matter have been described herein, such aspects need not be utilized in combination.

It is therefore intended that the appended claims (and/or any future claims filed in any corresponding application) cover all such changes and modifications that are within the scope of the claimed subject matter.

I claim:

1) A system operated by a user, the user having a user device, the system comprising:
at least one hardware processor;
a biosensor device operably connected to the user device, the biosensor device wearable by the user; and the at least one hardware processor performing a series of steps to manage health condition, the operation being a series of steps comprising:
monitoring for an irregularity;
identifying a symptom;
based on the symptom reporting the status of the symptom to the user;
offering an intervention based on an evaluation of the symptom in real-time;
providing the intervention to the user;
determining an efficacy of the intervention; and
improving the monitoring of the symptom of the user based on the learned information.

2) The system of claim 1, wherein the operations of the system further comprise the step of:
adapting the operations to improve the efficacy of the intervening with the individual.

3) The system of claim 2, wherein the determining the efficacy of the intervening with the individual comprises querying the user with an interaction with the system where the interaction is a dialog interaction, typing, interacting with the display, voice interaction, haptic feedback, menu interacting, or other interaction with the system.

4) The system of claim 1, wherein a networked computational server of the system comprises the at least one hardware processor, wherein the system comprises a biosensor device operably connected to the networked computational server.

5) The system of claim 4, wherein the user device operably connects to the networked computational server and the biosensor device.

6) The system of claim 1, wherein the operations further comprise:
reporting a status of the individual to a third party.

7) The system of claim 1, wherein the system comprises a biosensor configured to produce one or more signals that relate to the biological property, wherein the biological property is selected from a group consisting essentially of: a heart rate (HR), a heart rate variability (HRV), an oxygen saturation (Sp02), an electrodermal activity (EDA), a breathing rate (BR), a movement, a body temperature (BT), a blood sugar level, a perspiration level, or a body metric, and any combination thereof.

8) The system of claim 1, wherein the system comprises a camera system to image the individual and produce one or more signals that relate to the biological property based on a processing of one or more images of the individual.

9) The system of claim 1, wherein the intervention to alleviate the symptom comprises a therapy.

10) The system of claim 9, wherein the therapy is selected from a group consisting essentially of: engaging in cognitive-behavioral therapy (CBT), engaging in dialectical behavior therapy (DBT), engaging in acceptance and commitment therapy (ACT), engaging in a positive activity, engaging in a breathing exercise, general therapy and any combination thereof.

11) A system operated by a user, the user having a user device, the system comprising:
at least one hardware processor;
a biosensor device operably connected to the user device, the biosensor device wearable by the user; and [symbol] steps comprising:
monitoring for an irregularity;
identifying a symptom;
evaluating the symptom; and
based on the symptom the system determining a context of the symptom, correlating the symptom, the context, the intervention, and the efficacy so as to improve the monitoring for the symptoms of the user based on the learned information.

12) The system of claim 1, wherein the operations of the system further comprise:
correlating the symptom with a context which corresponds with onset of the symptom.

13) The system of claim 4, wherein the operations further comprise:
detecting the context in a future of the individual; and
intervening with the individual to prevent or mitigate the symptom.

14) The system of claim 11, wherein the operations of the system further comprise the step of:
adapting the operations to improve the efficacy of the intervening with the individual.

15) The system of claim 14, wherein the determining the efficacy of the intervening with the individual comprises querying the user with an interaction with the system where the interaction is an dialog interaction, typing, interacting with the display, voice interaction, haptic feedback, menu interacting, or other interaction with the system.

16) A system operated by a user device, the system comprising:
at least one hardware processor;
a biosensor device operably connected to the user device; and
the at least one hardware processor performing a series of steps to manage health condition, the an operation being a series of steps comprising:
monitoring for an irregularity;
identifying a symptom;
based on the symptom reporting the status of the symptom to the user;
offering an intervention based on an evaluation of the symptom in real-time;
providing the intervention to the user;
determining an efficacy of the intervention; and
improving the monitoring of the symptom of the user based on the learned information.

17) The system of claim 16 wherein the biosensor device is wearable by a user.

18) The system of claim 16 wherein the biosensor device is spaced apart from a user.

19) The system of claim 16 wherein the hardware processor and the biosensor device are integrated together in a single unit.

20) The system of claim 16, wherein the operations of the system further comprise the step of:
adapting the operations to improve the efficacy of the intervening with the individual.
